(19) United States
(12) Patent Application Publication
(10) Pub. No.: US 2004/0006061 A1
(43) Pub. Date: Jan. 8, 2004

(54) Alkoxybenzylamine

(76) Inventors: Wolfgang Haap, Grenzach-Wyhlen (DE); Werner Holzl, Eschentzwiller (FR); Karin Petzold, Fischingen (DE)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/263,445

(22) Filed: Oct. 2, 2002

(30) Foreign Application Priority Data
Oct. 4, 2001 (EP) 01810970.2

(51) Int. Cl. A61K 31/55; A61K 31/445; C07D 211/70

(52) U.S. Cl. 514/212.01; 514/317; 514/649; 514/408; 514/357; 540/484; 546/236; 548/578; 564/336; 546/329

(57) ABSTRACT

There are described alkoxybenzylamines corresponding to formula

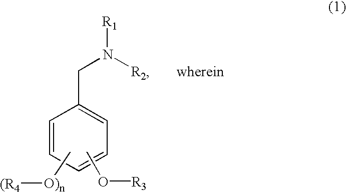

R₁ and R₂ are each independently of the other hydrogen; C₁-C₂₀alkyl, C₂-C₂₀alkenyl, C₂-C₂₀alkynyl, C₃-C₁₂cycloalkyl, C₃-C₇cycloalkyl-C₁-C₂₀alkyl, C₁-C₆alkoxy-C₁-C₂₀alkyl, C₁-C₁₂alkoxycarbonyl, phenyl or phenyl-C₁-C₂₀alkyl, each of which is unsubstituted or substituted by one or more of hydroxy, halogen, C₁-C₁₈alkyl, C₁-C₁₈alkoxy or amino; carboxy; or pyridino-C₁-C₅alkyl; or

R₁ and R₂, together with the nitrogen atom linking them, form a 5- to 7-membered monocyclic heterocyclic ring;

R₃ and R₄ are each independently of the other C₁-C₂₀alkyl which is unsubstituted or substituted by one or more of hydroxy, halogen, C₁-C₁₈alkyl, C₁-C₁₈alkoxy or amino; and

n is 0 or 1.

They are suitable for the antimicrobial treatment of surfaces, as antimicrobial active substances against gram-positive and gram-negative bacteria.

The present invention relates to alkoxybenzylamines, to the preparation of such compounds, and to their use for the antimicrobial treatment of surfaces, as antimicrobial active substances against gram-positive and gram-negative bacteria, yeasts and fungi and also for the preservation of cosmetics, household products, textiles and plastics and for use in disinfectants.

The alkoxybenzylamines according to the invention correspond to formula

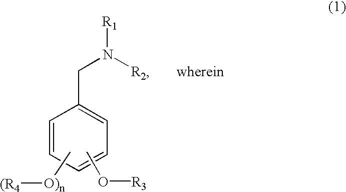

R₁ and R₂ are each independently of the other hydrogen; C₁-C₂₀alkyl, C₂-C₂₀alkenyl, C₂-C₂₀alkynyl, C₃-C₁₂cycloalkyl, C₃-C₇cycloalkyl-C₁-C₂₀alkyl, C₁-C₆alkoxy-C₁-C₂₀alkyl, C₁-C₁₂alkoxycarbonyl, phenyl or phenyl-C₁-C₂₀alkyl, each of which is unsubstituted or substituted by one or more of hydroxy, halogen, C₁-C₁₈alkyl, C₁-C₁₈alkoxy or amino; carboxy; or pyridino-C₁-C₅alkyl; or

R₁ and R₂, together with the nitrogen atom linking them, form a 5- to 7-membered monocyclic heterocyclic ring;

R₃ and R₄ are each independently of the other C₁-C₂₀alkyl which is unsubstituted or substituted by one or more of hydroxy, halogen, C₁-C₁₈alkyl, C₁-C₁₈alkoxy or amino; and

n is 0 or 1.

C₁-C₂₀Alkyl radicals are straight-chain or branched alkyl radicals, such as, for example, methyl, ethyl, n-propyl, isopropyl, n-butyl, sec-butyl, tert-butyl, amyl, isoamyl or tert-amyl, heptyl, octyl, isooctyl, nonyl, decyl, undecyl, dodecyl, tetradecyl, pentadecyl, hexadecyl, heptadecyl, octadecyl or eicosyl.

C₃-C₁₈Cycloalkyl is, for example, cyclopropyl, cyclobutyl, cyclopentyl, cycloheptyl, cyclooctyl, cyclononyl, cyclodecyl, cycloundecyl, cyclododecyl, cyclotetradecyl, cyclopentadecyl, cyclohexadecyl, cycloheptadecyl, cyclooctadecyl and especially cyclohexyl.

Alkenyl, within the context of the definitions above, is, inter alia, allyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₁-C₅Alkoxy radicals are straight-chain or branched radicals, such as, for example, methoxy, ethoxy, propoxy, butoxy, pentyloxy, heptyloxy, octyloxy, isooctyloxy, nonyloxy, decyloxy, undecyloxy, dodecyloxy, tetradecyloxy, pentadecyloxy, hexadecyloxy, heptadecyloxy, octadecyloxy or eicosyloxy.

Alkynyl includes, for example, ethynyl, propargyl, 2-butynyl, 1-pentenyl and 2-pentenyl.

Preference is given to compounds of formula (1) wherein

R₁ and R₂ are each independently of the other hydrogen; C₁-C₂₀alkyl, cyclo-C₅-C₇alkyl, phenyl or phenyl-C₁-C₄alkyl, each of which is unsubstituted or substituted by one or more of hydroxy, halogen, C₁-C₁₈alkyl, C₁-C₁₈alkoxy or amino; or pyridino-C₁-C₅alkyl.

Of those, special preference is given to compounds of formula (1) wherein

R₁ and R₂ are each independently of the other C₁-C₂₀alkyl; phenyl; phenyl-C₁-C₄alkyl; or pyridino-C₁-C₄alkyl;

and especially to such compounds of formula (1) wherein

R₁ and R₂ are each independently of the other C₁-C₁₂alkyl; benzyl; or pyridinoethyl.

Preference is given also to compounds of formula (1) wherein

R₁ and R₂, together with the nitrogen atom linking them, form a —(CH₂)₂₋₆— radical that is not further substituted or that is substituted by one or more C₁-C₅alkyl groups, and that is optionally interrupted by one or two —O— and/or —NR′— groups and/or by

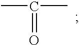

R′ is hydrogen; C₁-C₁₈alkyl, cyclo-C₅-C₇alkyl or phenyl, each of which is unsubstituted or substituted by one or more of hydroxy, halogen, C₁-C₁₈alkyl, C₁-C₁₈alkoxy, amino or quaternary ammonium; or —COR″; and

R″ is hydrogen; or C₁-C₄alkyl.

Of those special preference is given to compounds of formula (1) wherein

R′ is hydrogen; C₁-C₅alkyl; a radical of formula

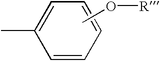

or a radical of formula

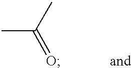

and

R′″ is hydrogen; or C₁-C₅alkyl;

and more especially to such compounds of formula (1) wherein

R₃ and R₄ are each independently of the other C₁-C₁₈alkyl.

Especially preferred are compounds of formula (1) wherein

R₃ is C₃-C₁₂alkyl, and such compounds wherein

R₄ is methyl.

Also preferred are compounds of formula (1) wherein R₃ and R₄ have identical meanings.

Compounds of formula

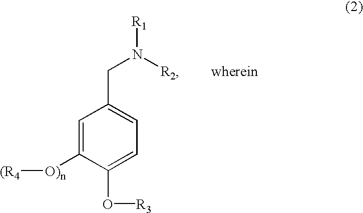

R₁, R₂, R₃, R₄ and n are as defined for formula (1), are especially preferred

Special preference is given to compounds of formula (2) wherein

R₁ and R₂, together with the nitrogen atom linking them, form a —(CH₂)₂₋₆— radical that is not further substituted or that is substituted by one or more C₁-C₅alkyl groups and that is optionally interrupted by one or two —O— and/or —NR′— groups and/or by

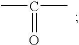

R′ is hydrogen; C₁-C₁₈alkyl, cyclo-C₅-C₇alkyl or phenyl, each of which is unsubstituted or substituted by one or more of hydroxy, halogen, C₁-C₁₈alkyl, C₁-C₁₈alkoxy, amino or quaternary ammonium; or —COR″; and

R″ is hydrogen; or C₁-C₄alkyl;

R₃ and R₄ are each independently of the other C₁-C₁₈alkyl; and

n is 0; or 1.

Preference is given furthermore to compounds of formula (2) wherein

R₁ and R₂ are each independently of the other C₁-C₂₀alkyl; phenyl; phenyl-C₁-C₄alkyl; or pyridino-C₁-C₄alkyl;

R₃ is C₃-C₁₂alkyl; and

R₄ is C₁-C₁₂alkyl, especially methyl.

Further alkoxybenzylamines according to the invention are listed by way of example in the following Table 1:

TABLE 1

| Compound | | Purity | MIC [μg/ml] | |
|---|---|---|---|---|
| of formula | Structure | [% by area] | SH | EC |
| (3) | 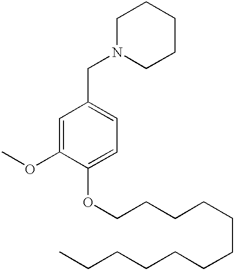 | 90 | 3.75 | >120 |
| (4) | 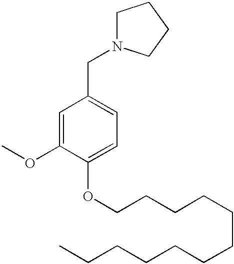 | 96 | 3.75 | 30 |
| (5) | 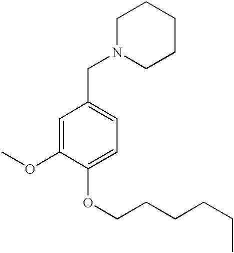 | 100 | >120 | >120 |
| (6) | 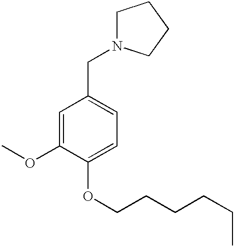 | 95 | >120 | >120 |
| (7) | 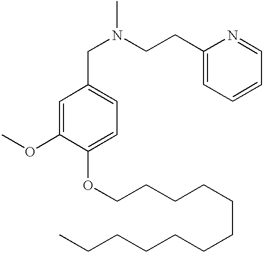 | 13.1 | 60 | >120 |
| (8) | 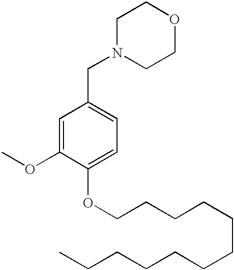 | 69 | >120 | >120 |
| (9) | 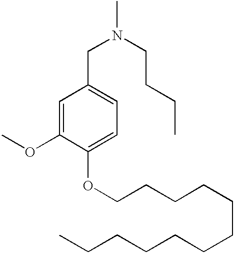 | 93.2 | 15 | >120 |
| (10) | 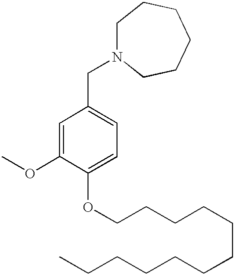 | 95.2 | 3.75 | >120 |
| (11) | 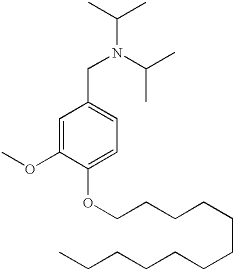 | 87 | >120 | >120 |
| (12) | 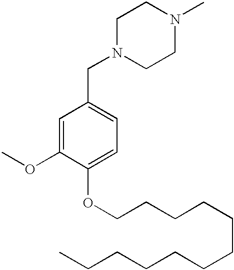 | 85 | 3.75 | 120 |
| (13) | 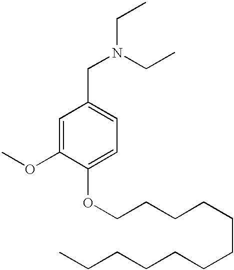 | 89 | 3.75 | >120 |
| (14) | 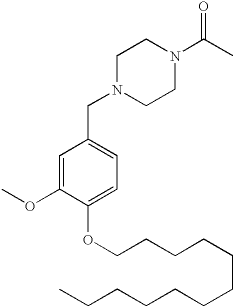 | 84 | >120 | >120 |
| (15) | 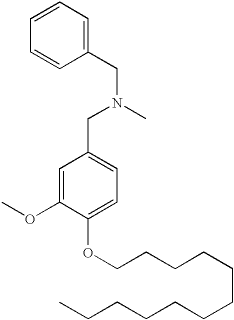 | 95 | 15 | >120 |
| (16) | 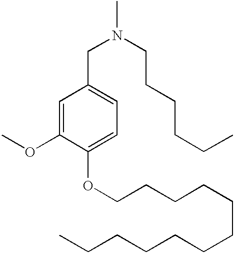 | 95 | 120 | >120 |
| (17) | 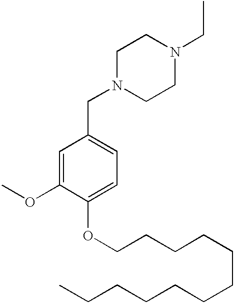 | 55 | 7.5 | >120 |
| (18) | 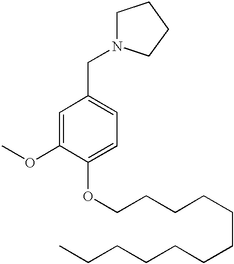 | 82 | 3.75 | 15 |
| (19) | 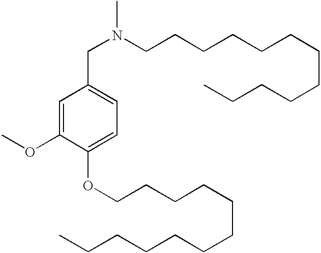 | 60 | >120 | >120 |
| (20) | 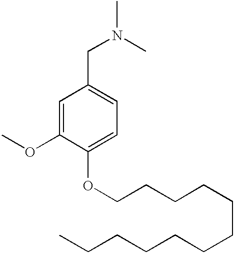 | 75 | 7.5 | 15 |
| (21) | 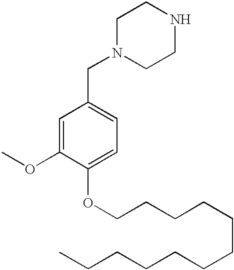 | — | >120 | >120 |
| (22) | 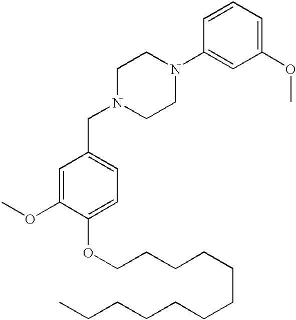 | 8 | >120 | >120 |
| (23) | 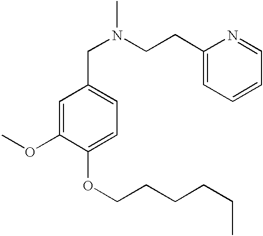 | 11 | 60 | >120 |
| (24) | 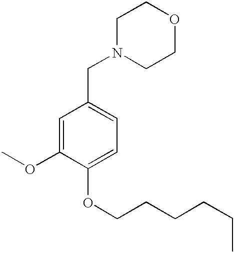 | 25 | >120 | >120 |
| (25) | 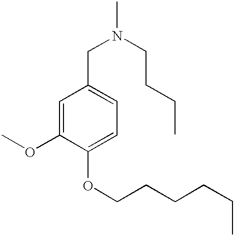 | 69 | >120 | >120 |
| (26) | 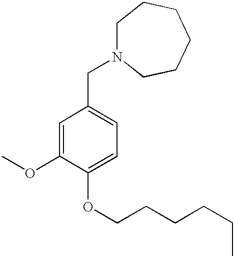 | — | >120 | >120 |
| (27) | 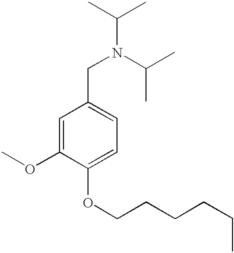 | 92 | >120 | >120 |
| (28) | 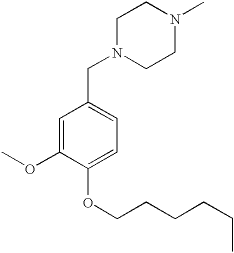 | 60 | >120 | >120 |
| (29) | 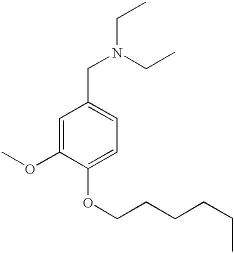 | 90 | >120 | >120 |
| (30) | 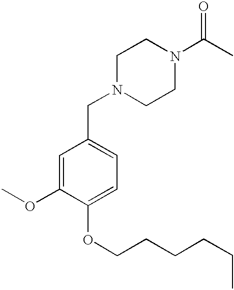 | 74 | >120 | >120 |
| (31) | 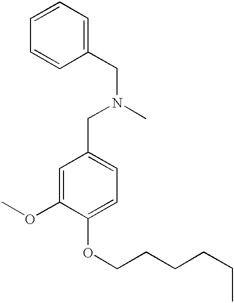 | 90 | >120 | >120 |
| (32) | 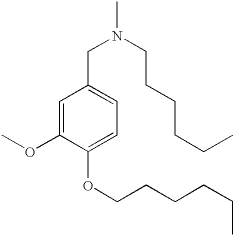 | 93 | 60 | 7.5 |
| (33) | 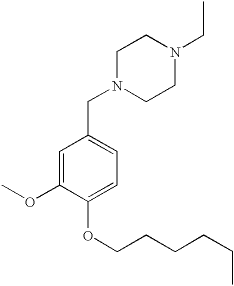 | 87 | >120 | >120 |
| (34) | 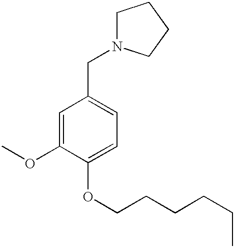 | 85 | >120 | >120 |
| (35) | 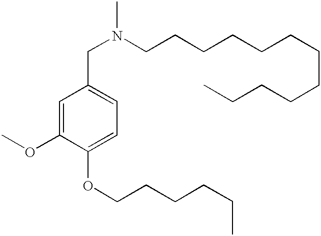 | 94 | 15 | >120 |
| (36) | 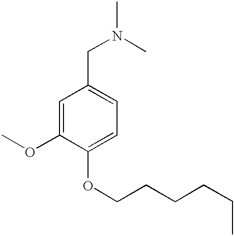 | 84 | >120 | >120 |
| (37) | 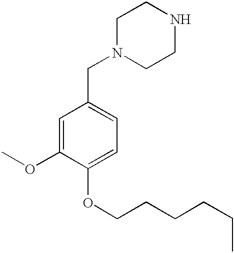 | 12 | >120 | >120 |
| (38) | 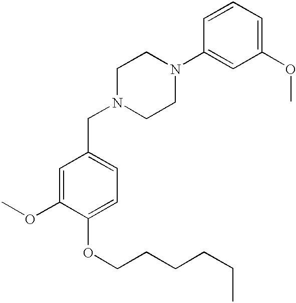 | 9 | >120 | >120 |
| (39) | 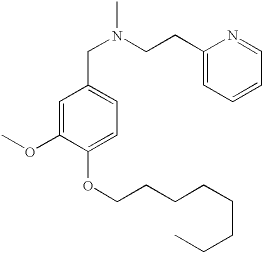 | 1 | 120 | >120 |
| (40) | 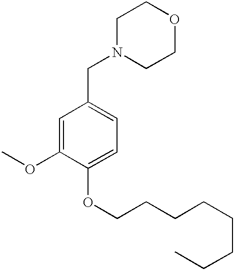 | 63 | >120 | >120 |
| (41) | 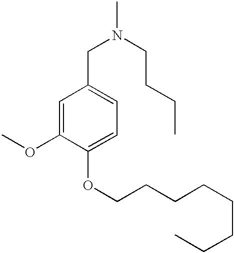 | 89 | 15 | 15 |
| (42) | 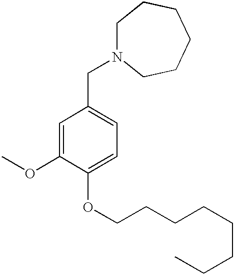 | 88 | 15 | 15 |
| (43) | 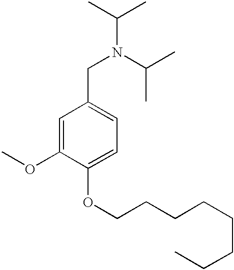 | 61 | >120 | >120 |
| (44) | 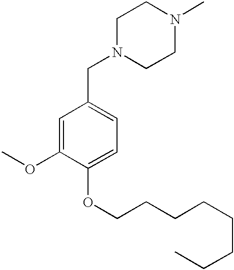 | 55 | 120 | 120 |
| (45) | 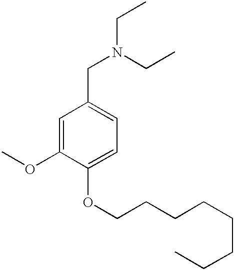 | 81 | 120 | 120 |
| (46) | 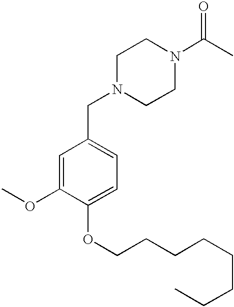 | 69 | >120 | 120 |
| (47) | 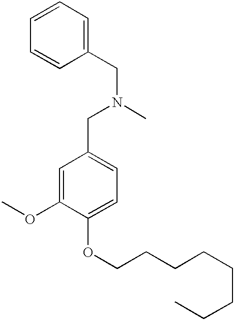 | 88 | >120 | >120 |
| (48) | 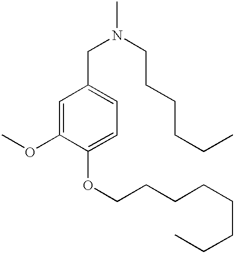 | 85 | 120 | >120 |
| (49) | 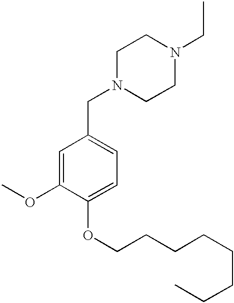 | 48 | 60 | 120 |
| (50) | 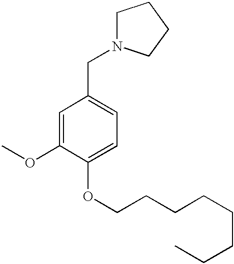 | 94 | 60 | 60 |
| (51) | 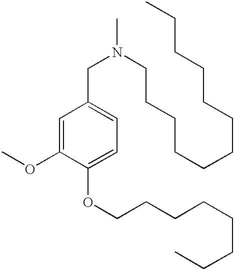 | 82 | >120 | >120 |
| (52) | 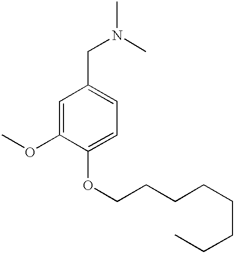 | 81 | 120 | 120 |
| (53) | 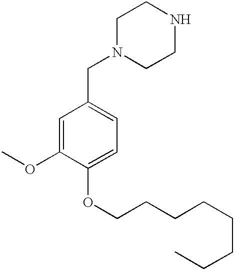 | — | >120 | >120 |
| (54) | 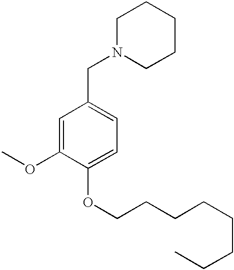 | 95 | 60 | 30 |
| (55) | 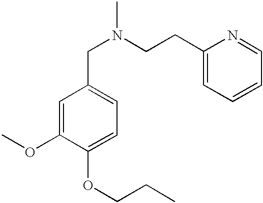 | — | >120 | >120 |
| (56) | 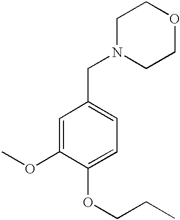 | 26 | 120 | >120 |
| (57) | 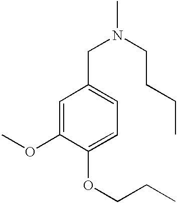 | 80 | >120 | 120 |
| (58) | 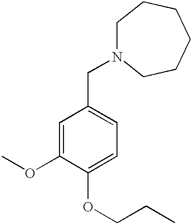 | 85 | 120 | >120 |
| (59) | 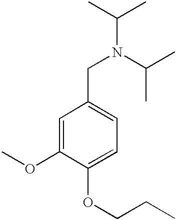 | — | >120 | >120 |
| (60) | 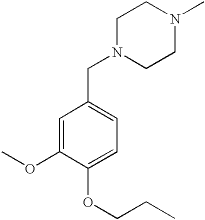 | 62 | >120 | >120 |
| (61) | 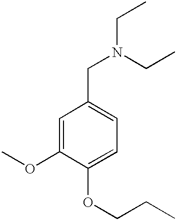 | 81 | >120 | >120 |
| (62) | 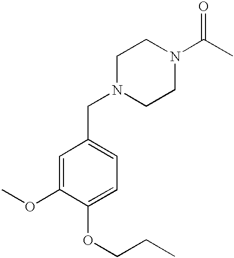 | 79 | >120 | >120 |
| (63) | 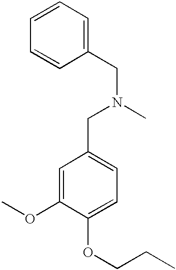 | 86 | >120 | >120 |
| (64) | 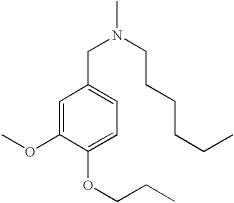 | 97 | >120 | >120 |
| (65) | 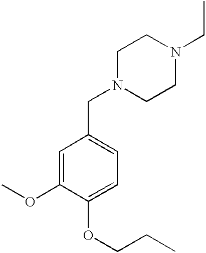 | 92 | >120 | >120 |
| (66) | 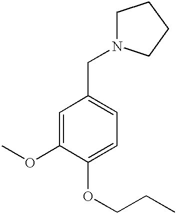 | 93 | >120 | >120 |
| (67) | 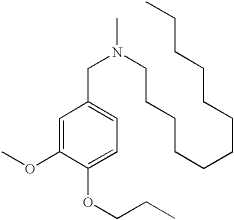 | 86 | 60 | >120 |
| (68) | 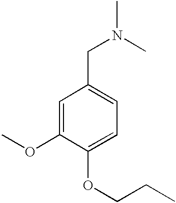 | — | >120 | >120 |
| (69) | 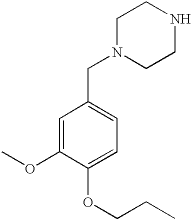 | — | >120 | >120 |
| (70) | 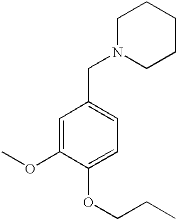 | 93 | >120 | >120 |
| (71) | 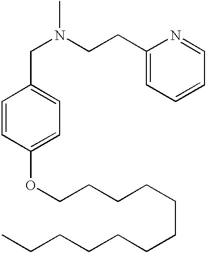 | 1 | >120 | >120 |
| (72) | 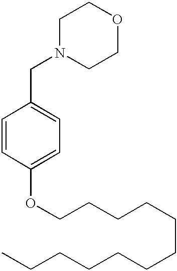 | 23 | >120 | >120 |
| (73) | 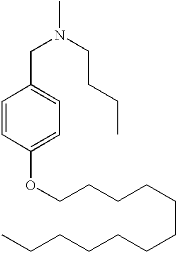 | 72 | 120 | >120 |
| (74) | 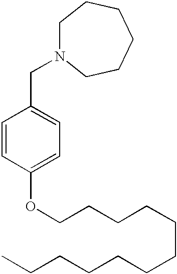 | 85 | 3.75 | >120 |
| (75) | 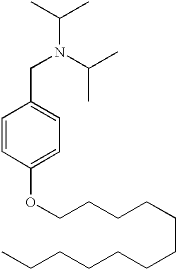 | 58 | >120 | >120 |
| (76) | 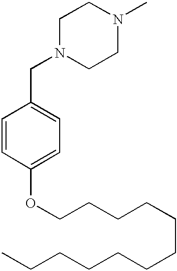 | 84 | 7.5 | >120 |
| (77) | 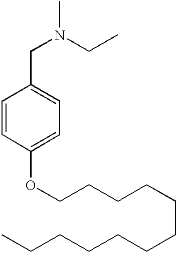 | 93 | 15 | >120 |
| (78) | 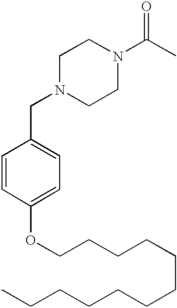 | 83 | >120 | >120 |
| (79) | 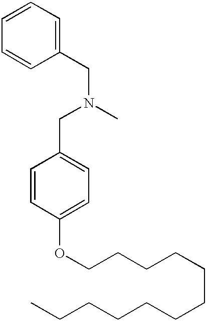 | 79 | 60 | >120 |
| (80) | 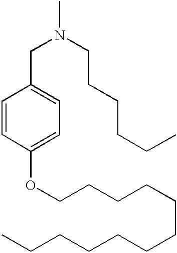 | 66 | 60 | >120 |
| (81) | 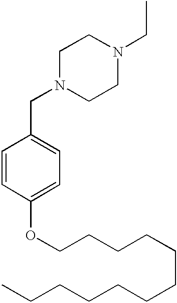 | — | 15 | >120 |
| (82) | 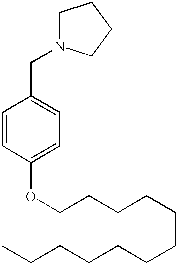 | 96 | 7.5 | 60 |
| (83) | 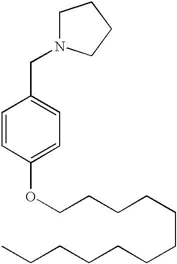 | — | >120 | >120 |
| (84) | 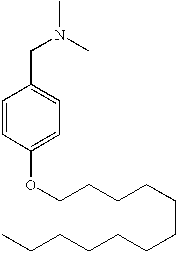 | 66 | 7.5 | 60 |
| (85) | 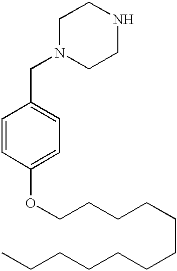 | — | >120 | >120 |
| (86) | 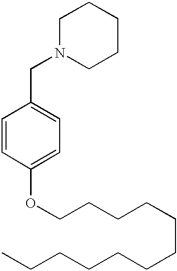 | 73 | 7.5 | >120 |
| (87) | 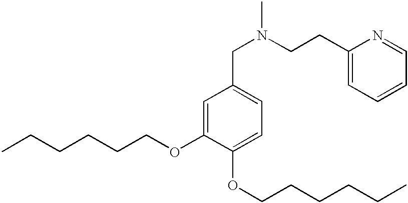 | 15 | 30 | >120 |
| (88) | 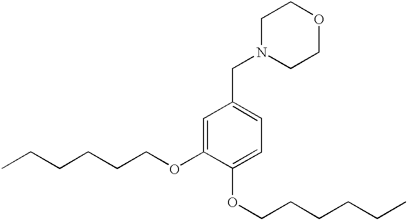 | 73 | >120 | >120 |
| (89) | 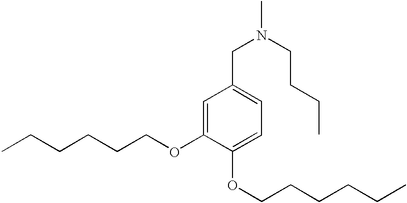 | 78 | 60 | >120 |
| (90) | 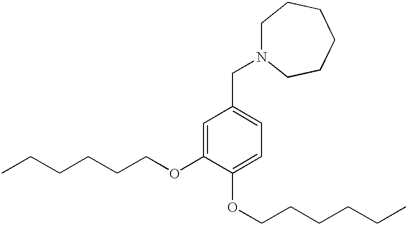 | 77 | 7.5 | >120 |
| (91) | 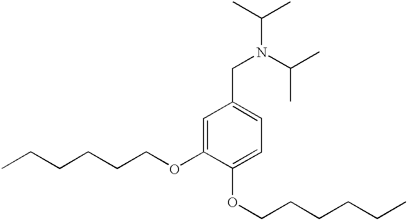 | 63 | >120 | >120 |
| (92) | 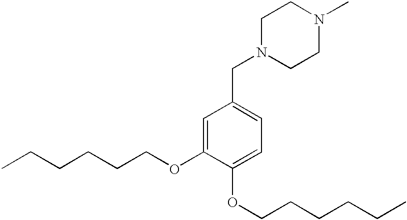 | 90 | 15 | 60 |
| (93) | 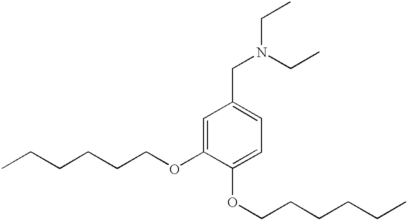 | 97 | 7.5 | 120 |
| (94) | 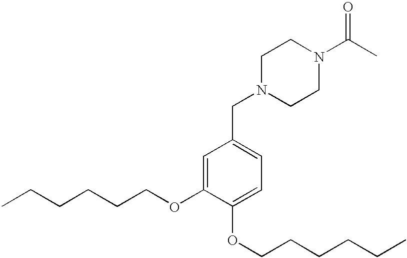 | 83 | >120 | >120 |
| (95) | 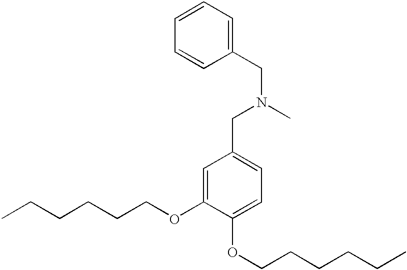 | 77 | >120 | >120 |
| (96) | 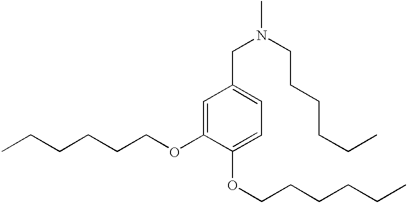 | 87 | 120 | >120 |
| (97) | 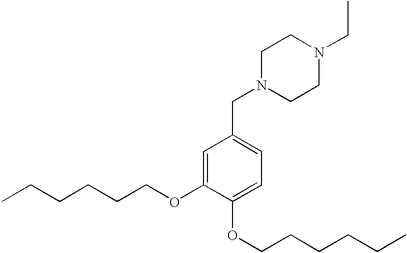 | 89 | 30 | 120 |
| (98) | 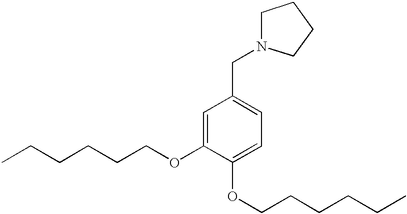 | 97 | 3.75 | 7.5 |
| (99) | 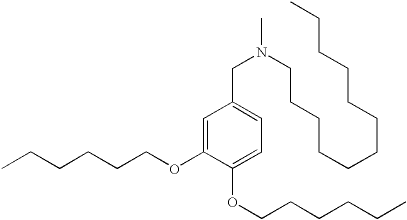 | 62 | >120 | >120 |
| (100) | 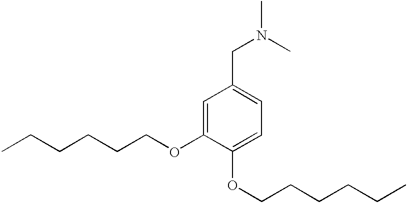 | 96 | 7.5 | 7.5 |
| (101) | 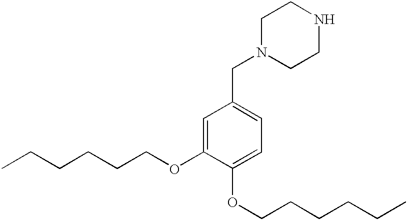 | — | >120 | >120 |
| (102) | 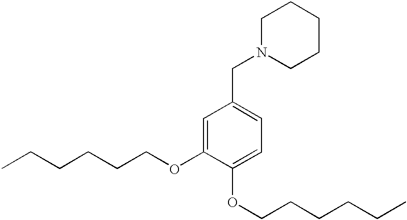 | 92 | 7.5 | >120 |

SH = Staphylococcus hominis
EC = Escherichia coli

Determination of the Minimum Inhibitory Concentration (MIC Value) in Microtitre Plates

Nutrient medium:

Casein-soybean flour-peptone broth for preparation of pre-cultures of test bacteria and yeast.

Test organisms used:

Staphylococcus hominis DSM 20328

Escherichia coli NCTC 8196

Procedure:

The test substances are pre-dissolved in dimethyl sulfoxide (DMSO) and tested in a dilution series of 1:2.

The bacteria are cultured overnight in CASO broth and washed off using 10 ml of 0.85% sodium chloride solution (+0.1% Triton X-100).

All the test organisms are adjusted to an organism count of 1-5×10⁶ CFU/ml using 0.85% sodium chloride solution.

The test substances are pre-pipetted into microtitre plates in amounts of 8 μl per well.

Pre-diluted test organism suspensions are diluted 1:100 in CASO broth.

The test batches are incubated for 48 hours at 37° C.

After incubation, the growth is determined on the basis of the turbidity of the test batches (optical density) at 620 nm in a microplate reader.

The minimum inhibitory concentration (MIC value) is the concentration of substance at which there is found (compared to the growth of the control) an appreciable inhibition of growth (≦20% growth) of the test organisms.

One microtitre plate is used for each test organism and substance concentration.

All the substances are tested in duplicate.

The alkoxybenzylamines according to the invention are prepared by customary methods of synthesis. In a first reaction step, an alkoxyhydroxybenzaldehyde, a hydroxybenzaldehyde or a dihydroxybenzaldehyde is alkylated with the appropriate alkyl halide in a suitable solvent, for example toluene, xylene, DMF or THF, over a period of from 1 to 24 hours at a temperature of from 40 to 130° C., using an auxiliary base, for example a sodium alkanolate (methanolate, ethanolate, tert-butanolate), soda, potash or sodium hydroxide, to form the corresponding mono- or bis-alkoxybenzaldehyde. The corresponding mono- or bis-alkoxybenzaldehyde is then condensed with a secondary amine in a suitable solvent, for example THF, dioxane or toluene, or in an excess of the secondary amine without solvent by means of a water-removing agent, for example trimethyl orthoformate or molecular sieve, or by removal of water by azeotropic distillation. By simultaneous addition of a reducing agent, for example sodium cyanoborohydride, sodium borohydride, lithium aluminium hydride or formic acid, the corresponding mono- or bis-alkoxybenzylamines are obtained directly in the course of from 0.5 to 24 hours at a temperature of from −10 to 120° C.

The complete course of the reaction can be represented by the following scheme:

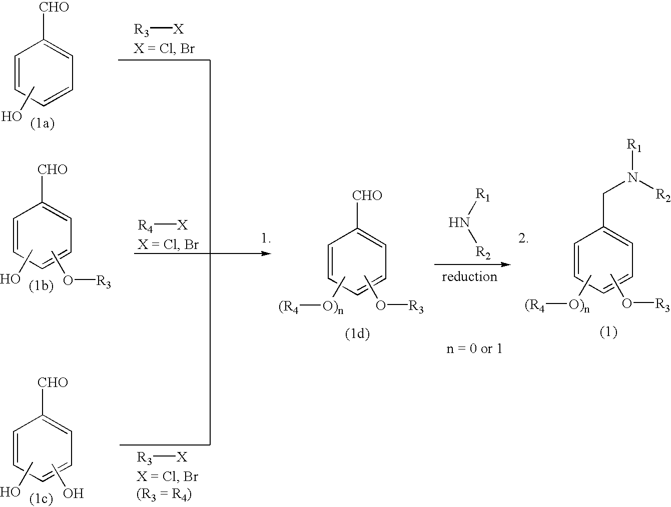

The alkoxybenzylamines used in accordance with the invention exhibit pronounced antimicrobial action, especially against pathogenic gram-positive and gram-negative bacteria and against bacteria of the skin flora, and also against yeasts and moulds.

They are accordingly suitable especially for disinfection, deodorisation, and for general and antimicrobial treatment of the skin and mucosa and of integumentary appendages (hair), more especially for the disinfection of hands and wounds.

They are accordingly suitable as antimicrobial active substances and preservatives in personal care preparations, for example shampoos, bath additives, haircare preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleaning cloths, oils or powders.

The invention accordingly relates also to a personal care preparation comprising at least one compound of formula (1) and cosmetically tolerable carriers or adjuvants.

The personal care preparation according to the invention contains from 0.01 to 15% by weight, preferably from 0.1 to 10% by weight, based on the total weight of the composition, of a compound of formula (1), and cosmetically tolerable adjuvants.

Depending upon the form of the personal care preparation, it comprises, in addition to the alkoxybenzylamine of formula (1), further constituents, for example sequestering agents, colorants, perfume oils, thickening or solidifying agents (consistency regulators), emollients, UV absorbers, skin protective agents, antioxidants, additives that improve the mechanical properties, such as dicarboxylic acids and/or aluminium, zinc, calcium or magnesium salts of C₁₄-C₂₂fatty acids, and, optionally, preservatives.

The personal care preparation according to the invention may be in the form of a water-in-oil or oil-in-water emulsion, an alcoholic or alcohol-containing formulation, a vesicular dispersion of an ionic or non-ionic amphiphilic lipid, a gel, a solid stick or an aerosol formulation.

As a water-in-oil or oil-in-water emulsion, the cosmetically tolerable adjuvant contains preferably from 5 to 50% of an oil phase, from 5 to 20% of an emulsifier and from 30 to 90% water. The oil phase may comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations according to the invention are used in various fields. There come into consideration, for example, especially the following preparations:

skin-care preparations, e.g. skin-washing and cleansing preparations in the form of tablet-form or liquid soaps, synthetic detergents or washing pastes,

bath preparations, e.g. liquid (foam baths, milks, shower preparations) or solid bath preparations, e.g. bath cubes and bath salts;

skin-care preparations, e.g. skin emulsions, multi-emulsions or skin oils;

cosmetic personal care preparations, e.g. facial make-up in the form of day creams or powder creams, face powder (loose or pressed), rouge or cream make-up, eye-care preparations, e.g. eyeshadow preparations, mascaras, eyeliners, eye creams or eye-fix creams; lip-care preparations, e.g. lipsticks, lip gloss, lip contour pencils, nail-care preparations, such as nail varnish, nail varnish removers, nail hardeners or cuticle removers;

intimate hygiene preparations, e.g. intimate washing lotions or intimate sprays;

foot-care preparations, e.g. foot baths, foot powders, foot creams or foot balsams, special deodorants and antiperspirants or callus-removing preparations;

light-protective preparations, such as sun milks, lotions, creams or oils, sun-blocks or tropicals, pre-tanning preparations or after-sun preparations;

skin-tanning preparations, e.g. self-tanning creams;

depigmenting preparations, e.g. preparations for bleaching the skin or skin-lightening preparations;

insect-repellents, e.g. insect-repellent oils, lotions, sprays or sticks;

deodorants, such as deodorant sprays, pump-action sprays, deodorant gels, sticks or roll-ons;

antiperspirants, e.g. antiperspirant sticks, creams or roll-ons;

preparations for cleansing and caring for blemished skin, e.g. synthetic detergents (solid or liquid), peeling or scrub preparations or peeling masks;

hair-removal preparations in chemical form (depilation), e.g. hair-removing powders, liquid hair-removing preparations, cream- or paste-form hair-removing preparations, hair-removing preparations in gel form or aerosol foams;

shaving preparations, e.g. shaving soap, foaming shaving creams, non-foaming shaving creams, foams and gels, preshave preparations for dry shaving, aftershaves or aftershave lotions;

fragrance preparations, e.g. fragrances (eau de Cologne, eau de toilette, eau de parfum, parfum de toilette, perfume), perfume oils or perfume creams;

dental care, denture-care and mouth-care preparations, e.g. toothpastes, gel toothpastes, tooth powders, mouthwash concentrates, anti-plaque mouthwashes, denture cleaners or denture fixatives;

cosmetic hair-treatment preparations, e.g. hair-washing preparations in the form of shampoos, hair conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile.

An antimicrobial soap has, for example, the following composition:

0.01 to 5% by weight of a compound of formula (1)

0.3 to 1% by weight titanium dioxide,

1 to 10% by weight stearic acid,

soap base ad 100%, e.g. a sodium salt of tallow fatty acid or coconut fatty acid, or glycerol.

A shampoo has, for example, the following composition:

0.01 to 5% by weight of a compound of formula (1),

12.0% by weight sodium laureth-2-sulfate,

4.0% by weight cocamidopropyl betaine,

3.0% by weight NaCl and

water ad 100%.

A deodorant has, for example, the following composition:

0.01 to 5% by weight of a compound of formula (1),

60% by weight ethanol,

0.3% by weight perfume oil, and

water ad 100%.

The invention relates also to an oral composition containing from 0.01 to 15% by weight, based on the total weight of the composition, of a compound of formula (1), and orally tolerable adjuvants.

Example of an oral composition:

10% by weight sorbitol,

10% by weight glycerol,

15% by weight ethanol,

15% by weight propylene glycol,

0.5% by weight sodium lauryl sulfate,

0.25% by weight sodium methylcocyl taurate,

0.25% by weight polyoxypropylene/polyoxyethylene block copolymer,

0.10% by weight peppermint flavouring,

0.1 to 0.5% by weight of a compound of formula (1), and

48.6% by weight water.

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name Olafluor.

The alkoxybenzylamines of formula (1) according to the invention are also suitable for treating, especially preserving, textile fibre materials. Such materials are undyed and dyed or printed fibre materials, e.g. of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose. Preferred suitable textile fibre materials are made of cotton.

The alkoxybenzylamines according to the invention are suitable also for treating, especially imparting antimicrobial properties to or preserving, plastics, e.g. polyethylene, polypropylene, polyurethane, polyester, polyamide, polycarbonate, latex etc.. Fields of use therefor are, for example, floor coverings, plastics coatings, plastics containers and packaging materials; kitchen and bathroom utensils (e.g. brushes, shower curtains, sponges, bathmats), latex, filter materials (air and water filters), plastics articles used in the field of medicine, e.g. dressing materials, syringes, catheters etc., so-called “medical devices”, gloves and mattresses.

Paper, for example papers used for hygiene purposes, may also be provided with antimicrobial properties using the alkoxybenzylamines according to the invention.

It is also possible for nonwovens, e.g. nappies/diapers, sanitary towels, panty liners, and cloths for hygiene and household uses, to be provided with antimicrobial properties in accordance with the invention. The alkoxybenzylamines of formula (1) are also used in washing and cleaning formulations, e.g. in liquid or powder washing preparations or softeners. The alkoxybenzylamines of formula (1) can also be used especially in household and general-purpose cleaners for cleaning and disinfecting hard surfaces.

A cleaning preparation has, for example, the following composition:

0.01 to 5% by weight of a compound of formula (1)

3.0% by weight octyl alcohol 4EO

1.3% by weight fatty alcohol C₈-C₁₀polyglucoside

3.0% by weight isopropanol

water ad 100%.

In addition to preserving cosmetic and household products, the preservation of technical products, the provision of technical products with antimicrobial properties and use as a biocide in technical processes are also possible, for example in paper treatment, especially in paper treatment liquors, printing ink thickeners of starch or cellulose derivatives, surface-coatings and paints.

The alkoxybenzylamines of formula (1) are also suitable for the antimicrobial treatment of wood and for the antimicrobial treatment of leather, the preserving of leather and the provision of leather with antimicrobial properties.

The compounds according to the invention are also suitable for the protection of cosmetic products and household products from microbial damage.

The following Examples illustrate, but do not limit, the present invention.

### IMPLEMENTATION EXAMPLES

### Preparation of Alkoxybenzylamines

General Method for the Preparation of Alkoxybenzaldehydes by Alkylation of Hydroxybenzaldehydes:

43 mmol of aldehyde are dissolved in 60 ml of ethanol and 6.5 g (49 mmol) of potassium carbonate are added. After the dropwise addition of a solution of 43 mmol of the appropriate bromoalkane in 30 ml of ethanol, heating at reflux is carried out for 16 hours. After filtering off over a small amount of silica gel and removing the solvent, the residue is taken up in 400 ml of tert-butyl methyl ether and the solution is washed three times with 200 ml of 1M sodium hydroxide solution each time. After removal of the solvent, the alkylated benzaldehydes are used for parallel amination without further purification.

Thus, for example, 10.0 g (73% of theory) of 3-methoxy-4-dodecyloxy-benzaldehyde are obtained from 6.5 g of vanillin and 10 ml of bromdodecane.

General Method for the Parallel Reductive Amination of Various Alkoxylated Benzaldehydes to Form the Compounds of Formulae (3) to (102)

1 mmol of the alkoxylated aldehyde is dissolved in 2.5 ml of trimethyl orthoformate (TMOF) and then a solution of 1 mmol of amine in 2.5 ml of TMOF and 0.19 ml of formic acid are added. After the addition of molecular sieve (4A), the mixture is stirred for 4 hours at 120° C. After concentration of the reaction mass, the product is purified by chromatography on silica gel (eluant ethanol/triethylamine 10/1). The purity and structure of the compounds are checked and confirmed by HPLC/MS. Thus, for example, by reaction of 3-methoxy-4-dodecyloxy-benzaldehyde and pyrrolidine, 85 mg (23% of theory) of the compound of formula (4) are obtained (m/z=375).

What is claimed is:

1. A compound of formula 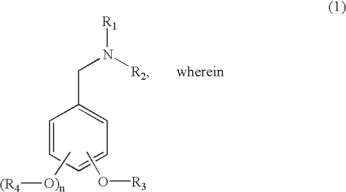
R₁ and R₂ are each independently of the other hydrogen; C₁-C₂₀alkyl, C₂-C₂₀alkenyl, C₂-C₂₀alkynyl, C₃-C₁₂cycloalkyl, C₃-C₇cycloalkyl-C₁-C₂₀alkyl, C₁-C₆alkoxy-C₁-C₂₀alkyl, C₁-C₁₂alkoxycarbonyl, phenyl or phenyl-C₁-C₂₀alkyl, each of which is unsubstituted or substituted by one or more of hydroxy, halogen, C₁-C₁₈alkyl, C₁-C₁₈alkoxy or amino; carboxy; or pyridino-C₁-C₅alkyl; or
R₁ and R₂, together with the nitrogen atom linking them, form a 5- to 7-membered monocyclic heterocyclic ring;
R₃ and R₄ are each independently of the other C₁-C₂₀alkyl which is unsubstituted or substituted by one or more of hydroxy, halogen, C₁-C₁₈alkyl, C₁-C₁₈alkoxy or amino; and
n is 0 or 1.

2. A compound according to claim 1, wherein
R₁ and R₂ are each independently of the other hydrogen; C₁-C₂₀alkyl, cyclo-C₅-C₇alkyl, phenyl or phenyl-C₁-C₄alkyl, each of which is unsubstituted or substituted by one or more of hydroxy, halogen, C₁-C₁₈alkyl, C₁-C₁₈alkoxy or amino; or pyridino-C₁-C₅alkyl.

3. A compound according to claim 1 , wherein
R₁ and R₂ are each independently of the other C₁-C₂₀alkyl; phenyl; phenyl-C₁-C₄alkyl; or pyridino-C₁-C₄alkyl.

4. A compound according to claim 1, wherein
R₁ and R₂ are each independently of the other C₁-C₁₂alkyl; benzyl; or pyridinoethyl.

5. A compound according to claim 1, wherein
R₁ and R₂, together with the nitrogen atom linking them, form a —(CH₂)₂₋₆— radical that is not further substituted or that is substituted by one or more C₁-C₅alkyl groups, and that is optionally interrupted by one or two —O— and/or —NR′— groups and/or by 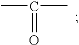
R′ is hydrogen; C₁-C₁₈alkyl, cyclo-C₅-C₇alkyl or phenyl, each of which is unsubstituted or substituted by one or more of hydroxy, halogen, C₁-C₁₈alkyl, C₁-C₁₈alkoxy, amino or quaternary ammonium; or —COR″; and
R″ is hydrogen; or C₁-C₄alkyl.

6. A compound according to claim 5, wherein
R′ is hydrogen; C₁-C₅alkyl; a radical of formula 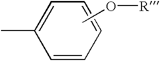
or a radical of formula 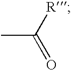
and
R″′ is hydrogen; or C₁-C₅alkyl.

7. A compound according to claim 1, wherein
R₃ and R₄ are each independently of the other C₁-C₁₈alkyl.

8. A compound according to claim 7, wherein
R₃ and R₄ have identical meanings.

9. A compound according claim 7, wherein
R₃ is C₃-C₁₂alkyl.

10. A compound according claim 7, wherein
R₄ is methyl.

11. A compound according to claim 1 that corresponds to formula 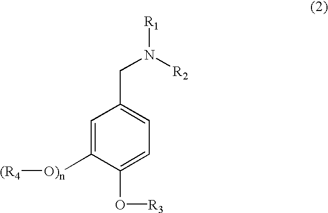
wherein
R₁, R₂, R₃, R₄ and n are as defined in claim 1.

12. A compound according claim 11, wherein
R₁ and R₂, together with the nitrogen atom linking them, form a —(CH₂)₂₋₆— radical that is not further substituted or that is substituted by one or more C₁-C₅alkyl groups and that is optionally interrupted by one or two —O— and/or —NR′— groups and/or by 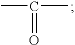
R′ is hydrogen; C₁-C₁₈alkyl, cyclo-C₅-C₇alkyl or phenyl, each of which is unsubstituted or substituted by one or more of hydroxy, halogen, C₁-C₁₈alkyl, C₁-C₁₈alkoxy, amino or quaternary ammonium; or —COR″; and
R″ is hydrogen; or C₁-C₄alkyl;
R₃ and R₄ are each independently of the other C₁-C₁₈alkyl; and
n is 0; or 1.

13. A compound according claim 11, wherein
R₁ and R₂ are each independently of the other C₁-C₂₀alkyl; phenyl; phenyl-C₁-C₄alkyl; or pyridino-C₁-C₄alkyl;
R₃ is C₃-C₁₂alkyl; and
R₄ is C₁-C₁₂alkyl, especially methyl.

14. A method of preparing a compound of formula (1) according to claim 1 by alkylation of a hydroxybenzaldehyde (when R₄ is hydrogen) or of an alkoxyhydroxyaldehyde or a dihydroxybenzaldehyde with an alkyl halide in a suitable solvent using an auxiliary base (first reaction step), and condensation of the resulting mono- or bis-alkoxybenzaldehyde of formula (1d) with a secondary amine and simultaneous addition of a reducing agent (second reaction step) to form the compound of formula (1), in a suitable solvent or in an excess of the secondary amine without solvent by means of a water-removing agent or by removal of water by azeotropic distillation, in accordance with the following reaction scheme:

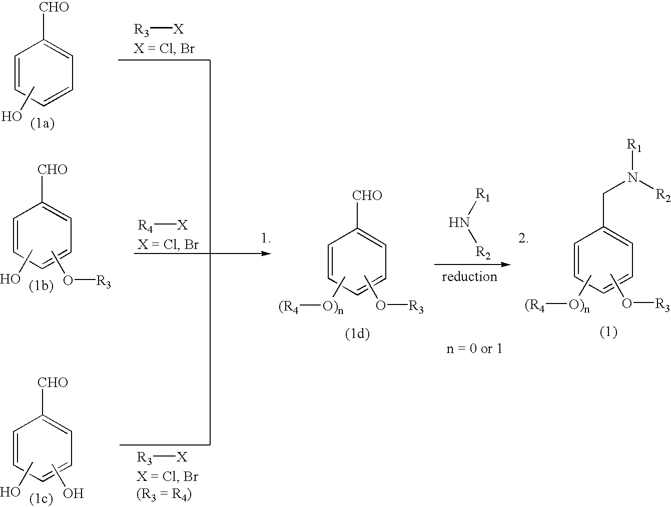

15. An antimicrobial method which comprises contacting a surface with a compound of formula 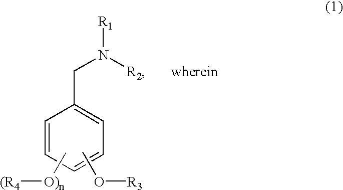
R₁ and R₂ are each independently of the other hydrogen; C₁-C₂₀alkyl, C₂-C₂₀alkenyl, C₂-C₂₀alkynyl, C₃-C₁₂cycloalkyl, C₃-C₇cycloalkyl-C₁-C₂₀alkyl, C₁-C₆alkoxy-C₁-C₂₀alkyl, C₁-C₁₂alkoxycarbonyl, phenyl or phenyl-C₁-C₂₀alkyl, each of which is unsubstituted or substituted by one or more of hydroxy, halogen, C₁-C₁₈alkyl, C₁-C₁₈alkoxy or amino; carboxy; or pyridino-C₁-C₅alkyl; or
R₁ and R₂, together with the nitrogen atom linking them, form a 5- to 7-membered monocyclic heterocyclic ring;
R₃ and R₄ are each independently of the other C₁-C₂₀alkyl which is unsubstituted or substituted by one or more of hydroxy, halogen, C₁-C₁₈alkyl, C₁-C₁₈alkoxy or amino; and
n is 0 or 1.

16. An antimicrobial method according to claim 15 for the treatment of skin, mucosa and hair which comprises applying an antimicobially effective amount of a compound of formula (1) as defined in claim 15 thereto.

17. A method according to claim 16, wherein the compound of formula (1) is used for disinfection and deodorisation.

18. An antimicrobial method according to claim 15 for the antimicrobial treatment of textile fibre materials which comprises applying an antimicobially effective amount of a compound of formula (1) as defined in claim 15 thereto.

19. A method according to claim 18, wherein the compound of formula (1) is used for preservation.

20. An antimicrobial method according to claim 15 for the antimicrobial treatment of washing and cleaning formulations which comprises applying an antimicobially effective amount of a compound of formula (1) as defined in claim 15 thereto.

21. A personal care preparation comprising
from 0.01 to 15% by weight, based on the total weight of the composition, of a compound of formula (1) according to claim 1, and cosmetically tolerable adjuvants.

22. An oral composition comprising from 0.01to 15% by weight, based on the total weight of the composition, of a compound of formula (1) according to claim 1, and orally tolerable adjuvants.
